(19) United States
(12) Patent Application Publication
(10) Pub. No.: US 2005/0201938 A1
(43) Pub. Date: Sep. 15, 2005

(54) Method for selectively inhibiting ghrelin action

(76) Inventors: Henry Uhlman Bryant, Indianapolis, IN (US); Mark Louis Heiman, Indianapolis, IN (US)

Correspondence Address: ELI LILLY AND COMPANY;PATENT DIVISION, P.O. BOX 6288, INDIANAPOLIS, IN, 46206-6288, US

(21) Appl. No.: 11/106,899

(22) Filed: Apr. 15, 2005

Related U.S. Application Data
(63) Continuation of application No. 10/258,259, filed on Oct. 18, 2002, now abandoned.
(60) Provisional application No. 60/204,897, filed on May 17, 2000.

(51) Int. Cl. C12Q 1/00; A61K 49/00; A61K 38/17

(52) U.S. Cl. 424/9.2; 435/4; 514/12

(57) ABSTRACT

The present invention provides a method selectively inhibiting ghrelin activity to treat a variety of diseases including obesity and related disorders, particularly in individuals who are genetically predisposed. One aspect of the invention comprises administering an agent that effectively neutralizes ghrelin. Another aspect comprises administering a ghrelin receptor (growth hor- mone secretagogue receptor) antagonist.

This application is a continuation of application Ser. No. 10/258,259 filed May 7, 2001, which was a national phase application under 35 USC §371 for PCT/US01/11752 filed on May 17, 2000, which claims priority of U.S. provisional Application No. 60/204,897 filed May 17, 2000.

The present invention is in the field of human medicine, particularly in the treatment of obesity and disorders associated with obesity such as diabetes mellitus. More specifically the invention relates to a method for treating obesity by administering a compound which blocks ghrelin action.

Obesity, and especially upper body obesity, is a common and very serious public health problem in the United States and throughout the world. According to recent statistics, more than 25% of the United States population and 27% of the Canadian population are overweight. Kuczmarski, Amer. J. of Clin. Nutr. 55: 495S-502S, 1992; Reeder et. al., Can. Med. Ass. J., 23: 226-233, 1992. Upper body obesity is the strongest risk factor known for type II diabetes mellitus, and is a strong risk factor for cardiovascular disease and cancer as well. Recent estimates for the medical cost of obesity are $150,000,000,000 worldwide. The problem has become serious enough that the surgeon general has begun an initiative to combat the ever-increasing adiposity rampant in American society.

Much of this obesity-induced pathology can be attributed to the strong association with dyslipidemia, hypertension, and insulin resistance. Many studies have demonstrated that reduction in obesity by diet and exercise reduces these risk factors dramatically. Unfortunately, these treatments are largely unsuccessful with a failure rate reaching 95%. This failure may be due to the fact that the condition is strongly associated with genetically inherited factors that contribute to increased appetite, preference for highly caloric foods, reduced physical activity, and increased lipogenic metabolism. This indicates that people inheriting these genetic traits are prone to becoming obese regardless of their efforts to combat the condition. Therefore, a means for effectively treating obese individuals, especially those who are genetically predisposed is needed.

The present invention provides a method of selectively inhibiting ghrelin activity in a mammal comprising administering to a mammal in need thereof a therapeutically-effective amount of a compound selected from the group consisting of a growth hormone secretagogue receptor antagonist (GHS-RA) and a ghrelin neutralizing agent (GNA). The invention further provides a method for treating obesity and related disorders in a mammal comprising administering to a mammal in need thereof a therapeutically-effective amount of a compound selected from the group consisting of a growth hormone secretagogue receptor antagonist (GHS-RA) and a ghrelin neutralizing agent (GNA). Other embodiments include in vitro and in vivo screening and assay methods.

Physiologists have postulated for years that, when a mammal overeats, the resulting excess fat signals to the brain that the body is obese which, in turn, causes the body to eat less and burn more fuel. G. R. Hervey, Nature 227: 629-631 (1969). This feedback model is supported by parabiotic experiments, which implicate circulating hormones that influence and regulate aspects of adiposity.

Growth hormone-releasing peptides (GHRPs) were first described in 1981 by Bowers and colleagues before the discovery of growth hormone-releasing hormone (GHRH). Momany F A, Bowers C Y, Reynolds G A, Chang D, Hong A, and Newlander K., Endocrinology 108: 31-39, 1981. Bowers C Y, Momany F A, Reynolds G A, Hong A., Endocrinology 114: 1537-1545 (1984). While Bowers' group demonstrated that such peptides could stimulate growth hormone (GH) release from isolated pituitary glands, they almost always reported a greater GH response when the GHRPs were administered in vivo. These data, reported in the early 1980's, suggested that such GHRPs have actions at both the hypothalamus and pituitary. After almost a decade, a non-peptidyl GH secretagogue (GHS) was reported and there have been many additional improvements in potency, bioavailability and Pharmacokinetics of GHS. Smith R G, Cheng K, Schoen W R, Pong S-S, Hickey G J, Jacks T M, Butler B S, Chan W W-S, Chaung L-Y P, Judith F, Taylor A M, Wyvratt Jr M J, and Fisher M H., Science 260: 1640-1643 (1993). A review of this general area was published recently. Smith R G, Van der Ploeg L H T, Howard A D, Feighner S D, Cheng K, Hickey G J, Wyvratt Jr M J, Fisher M H, Nargund R P, and Patchett A A., Endocrine Rev. 18:621-645 (1997).

After Smith and colleagues identified GHS, they isolated a GHS receptor (GHS-R) cDNA from both the pituitary and hypothalamus. Howard A D, Feighner S D, Cully D F, Arena J P, Liberator P A, Rosenblum C I, Hamelin M, Hreniuk D L, Palyha O C, Anderson J, Paress P S, Diaz C, Chou M, Liu K K, McKee K K, Pong S S, Chaung L Y, Elbrecht A, Dashkevicz M, Heavens R, Rigby M, Sirinathsinghji D J S, Dean D C, Melillo D G, Van der Ploeg L H T, Science 273: 974-977 (1996).

In December 1999, the endogenous ligand for GHS-R was identified and named ghrelin. Kojima M, Hosoda H, Date Y, Nakazato M, Matsuo H, Kangawa K., Nature 402: 656-60 (1999). They demonstrated that it is secreted by stomach tissue; and its mRNA is also expressed in the hypothalamus. Thus, the GHS-R now may be thought of as the ghrelin receptor. A review of this general area was recently published. Bowers C Y., J Clin. Endocrinol. Metab. 86: 1464-1469 (2001).

Although most GHS and GHRP studies were designed to exploit stimulation of the somatotropic axis, it has been demonstrated that these synthetic molecules induce sleep. Copinschi G, Leproult R, Vanonderbergen A, Caufriez A, Cole K Y, Schilling L M., Neuroendocrinol. 66: 278-286 (1997). Others have demonstrated that the synthetic GHS and GHRP also induce food intake. Locke W, Kirgis H D, Bowers C Y, and Abdo A A., Life Sci. 56:1347-1352 (1995). Okada K, Ishii S, Minami S, Sugihara H, Shibasaki T, and Wakabayashi I., Endocrinology 137:5155-5158 (1996). Moreover, Bennett et al. demonstrated that GHS-R is highly expressed in the arcuate nucleus. Bennett P A, Thomas G B, Howard A D, Feighner S D, Van der Ploeg L H T, Smith R G, and Robinson ICAF., Endocrinology 138: 4552-4557 (1997). In 1993, Dickson and colleagues observed an activation of such hypothalamic neurons after peripheral administration of a GHRP. Dickson S L, Leng G, and Robinson ICAF., Neuroscience 53: 303-306 (1993). Additionally, this group demonstrated that a majority of these activated neurons were those expressing neuropeptide-Y mRNA. Dickson S L and Luckman S M., Endocrinology 138: 771-777 (1997).

In view of this state of the art, the inventors of the presently claimed invention were most surprised when they demonstrated in an animal model that administration of ghrelin predominantly lead to fat deposition. Tschoep M., Smiley D L., and Heiman M L., Nature 407: 908-913 (2000). This lead them to postulate that ghrelin signals the CNS when energy homeostasis requires increased metabolic efficiency to induce energy preservation and a partitioning of fuel utilization from fat to carbohydrate to prevent hypoglycemia. Consequently, blocking or antagonizing ghrelin action compromises metabolic efficiency and induces energy consumption, primarily from fat stores.

Obesity, also called corpulence or fatness, is the excessive accumulation of body fat, usually caused by the consumption of more calories than the body uses. The excess calories are then stored as fat, or adipose tissue. Overweight, if moderate, is not necessarily obesity, particularly in muscular or large-boned individuals. In general, however, a body weight 20 percent or more over the optimum tends to be associated with obesity.

For purposes of the present invention, treating or treatment describes the management and care of a patient for the purpose of combating the disease, condition, or disorder. Treating includes the administration of a compound of present invention to prevent the onset of the symptoms or complications, alleviating the symptoms or complications, or eliminating the disease, condition, or disorder. Treating obesity therefore includes the inhibition of food intake, the inhibition of weight gain, and inducing weight loss in patients in need thereof.

For purposes of this invention, the term ‘related disorders’ includes but is not limited to type II diabetes, cardiovascular disease, cancer, and other disease states whose etiology stems from obesity.

The term ‘administering’ or ‘administration’ as used herein includes any means for introducing a GHS-RA or GNA into the body such that the substance is able to interact with the GHS-R or secreted ghrelin. Preferred routes of administration will introduce the substance into the systemic circulation. Examples include but are not limited to oral; transdermal; subcutaneous, intravenous, and intramuscular injection.

The active agents of the present invention are administered to a mammal, preferably a human, in accord with known methods, such as intravenous administration as a bolus or by continuous infusion over a period of time, by intramuscular, intraperitoneal, intracerebral, intracerobrospinal, subcutaneous, intra-articular, intrasynovial, intrathecal, intraocular, intralesional, oral, topical, inhalation or through sustained release.

A therapeutically-effective amount is at least the minimal dose, but less than a toxic dose, of an active agent which is necessary to impart therapeutic benefit to a mammal. Stated another way, a therapeutically-effective amount is an amount which induces, ameliorates or otherwise causes an improvement in the obese state of the mammal.

‘Carriers’ as used herein include pharmaceutically-acceptable carriers, excipients, or stabilizers which are nontoxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

The term ‘mammal’ as used herein refers to any animal classified as a mammal, including humans, domestic, farm and zoo animals, and sports or companion animals, etc. In a preferred embodiment of the invention, the mammal is a human.

The term ‘antibody’ is used in the broadest sense and specifically includes monoclonal antibodies, chimeric antibodies, humanized antibodies, and fully human antibodies.

The term ‘monoclonal antibody’ as used herein refers to an antibody obtained from a population of substantially homogeneous antibodies, i.e., the individual antibodies comprising the population are identical except for possible naturally-occurring mutations that may be present in minor amounts.

Antibody fragments means a portion of an intact antibody, preferably the antigen binding or variable region of the intact antibody. Examples of antibody fragments include Fab, Fab′, F(ab′)1 and Fv fragments; diabodies; linear antibodies (Zapata et al., Protein Engin. S(10): 1057-1 062 (1991)); single-chain antibody molecules; and multispecific antibodies formed from antibody fragments.

The term ‘Fv’ is the minimum antibody fragment, which contains a complete antigen-recognition and binding site. This region consists of a dimer of one heavy- and one light chain variable domain in tight, non-covalent association. It is in this configuration that the three complementarity-determining regions (CDRs) of each variable domain interact to define an antigen-binding site on the surface of the VH-VL dimer. Collectively, the six CDRs confer antigen-binding specificity to the antibody. However, even a single variable domain (or half of an Fv comprising only three CDR specific for an antigen) has the ability to recognize and bind antigen, although at a lower avidity than a complete antibody.

The Fab fragment also contains the constant domain of the light chain and the ‘first constant domain (CHI) of the heavy chain. Fab fragments differ from Fv fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. F(ab′)_{z} antibody fragments originally were produced as pairs of Fab′ fragments which have hinge cysteines between them. Other chemical couplings of antibody fragments are also known.

Papain digestion of antibodies produces two identical antigen-binding fragments, called Fab fragments, each with a single antigen-binding site, and a residual Fc fragment, a designation reflecting the ability to crystallize readily. Pepsin treatment yields an F(ab′)₂ fragment that has two antigen-combining sites and is still capable of cross-linking antigen.

The ‘light chains’ of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA and IgA2.

‘Single-chain Fv’ antibody fragments comprise the VH and VL domains of antibody, wherein these domains are present in a single polypeptide chain. Preferably, the Fv polypeptide further comprises a polypeptide linker between the VH and VL domain, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-3 15, 1994. As used herein, the term ‘immunoadhesion’ designates antibody-like molecules that combine the binding specificity of a heterologous protein (an ‘adhesion’) with the effector functions of immunoglobulin constant domains. Structurally, the immunoadhesions comprise a fusion of an amino acid sequence with the desired binding specificity which is other than the antigen recognition and binding site of an antibody (i.e., is heterologous), and an immunoglobulin constant domain sequence. The adhesion part of an immunoadhesion molecule typically is a contiguous amino acid sequence comprising at least the binding site of a receptor or a ligand. The immunoglobulin constant domain sequence in the immunoadhesion may be obtained from any immunoglobulin, such as IgG-1, IgG-2, IgG-3 or IgG-4 subtypes, IgA (including IgG-1 and IgA-2), IgE, IgD or IgM.

The term ‘diabodies’ refers to small antibody fragments with two antigen-binding sites, which fragments comprise a heavy-chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen binding sites. Diabodies are described more fully in, for example, EP 404.097, WO 93/1 1161; and Hollinger et al., Proc. NatI. Acad. Sci. USA 90: 6444-6448 (1993).

A GHS-RA is any compound that partially or fully antagonizes, blocks, or otherwise inhibits the biological action of ghrelin by binding to the GHS-R without stimulating the release of growth hormone. Therefore GHS (compounds that bind the GHS-R and stimulate the release of GH) are not consistent with the claimed method.

GHS-RA are compounds useful in the presently claimed method and include but are not limited to natural products, synthetic organic compounds, peptides, proteins, antibodies, antibody fragments, single chain antibodies, and antibody based constructs.

The current level of skill in the art of receptor binding and growth hormone assays places GHS-RAs well within the grasp of the ordinarily skilled artisan. There are several routine approaches for identifying a GHS-R. One basic scheme involves a receptor binding assay followed by a GH release assay. In this scheme, the GHS-RA test compound is first checked to determine if it binds GHS-R. This is accomplished using routine radiometric binding methods. Alternatively, a second messenger reporter such as calcium can be used to determine binding. One such assay is described in Kojima et al., Nature 402: 656-60 (1999).

Compounds that bind GHS-R are then exposed to primary pituitary cells, for example, and release of growth hormone is determined using standard commercially available assays. Compounds that bind but do not stimulate the release of GH should then be assayed for ghrelin antagonism by exposing pituitary cells to the GHS-RA in the presence of ghrelin and then assaying for GH release.

Antibody-based GHS-RAs are also consistent with the claimed method. Anti-GHS-R antibodies may be generated by a variety of well-known methods that include traditional antisera production and monoclonal antibody techniques. Modified antibody forms described above may then be produced using established techniques. Once generated, the antibodies are checked for GHS-RA activity in the manner described above.

Ghrelin neutralizing agents (GNAs) represent another aspect of the invention. In this embodiment, ghrelin is neutralized or otherwise rendered biologically inactive apart from the receptor. Agents suitable for this application are those which specifically bind ghrelin, preferably with a higher affinity constant than the GHS-R.

Antibody or antibody-based agents are preferred because they can be purposefully generated using well established techniques. Kojima et al., Nature 402: 656-60 (1999). Immunoadhesions (Fc fusion constructs, similar to Enbrel®, where the soluble ligand-binding domain of the GHS-R is fused to a human Fc) are also consistent with this aspect of the invention.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy.

In another embodiment of the invention, an article of manufacture containing materials useful in the presently claimed methods is provided. The article of manufacture comprises a container and a label. Suitable containers include, for example, bottles, vials, syringes, and test tubes. The containers may be formed from a variety of materials such as glass or plastic. The container holds a composition which is effective for specifically inhibiting ghrelin action and may have a sterile access port (for example the container may be an intravenous solution bag or a vial having a stopper pierceable by a hypodermic injection needle). The active agent in the composition is a GHS-RA and/or a GNA. The label on, or associated with, the container indicates that the composition is used for treating obesity and/or related disorders. The article of manufacture may further comprise a second container comprising a pharmaceutically-acceptable buffer, such as phosphate-buffered saline, Ringer's solution and dextrose solution. It may further include other materials desirable from a commercial end user standpoint, including other buffers, diluents, filters, needles, syringes, and package inserts with instructions for use.

The following examples are offered for illustrative purposes only, and are not intended to limit the scope of the present invention in any way.

### EXAMPLE 1

### Ghrelin Synthesis

Rat ghrelin was synthesized on an Advanced ChemTech® 396 synthesizer with FMOC amino acids and 50 minute diisopropylcarbodiimide (DIC)/1-hydroxibenzotriazole (HOBT) activated double couplings. FMOC-SER(Trt) was used in the couplings for Ser3. Following trityl deprotection using 1% TFA/5% tri-isopropylsilane in methylene chloride (DCM), the Ser3-hydroxyl was acylated using excess octanoic acid and 1,3[(Dimethylamino)propyl]-3-ethylcarbodiimide hydrochloride salt (EDAC) in the presence of 4-dimethylaminopyridine (DMAP). After removal of the N-terminal FMOC, a 2 hours cleavage was run using Reagent K. The precipitated peptide was washed with ethyl ether and dried in vacuo. The material was dissolved in aqueous acetic acid and purified over a 2.2×25 cm VydacC18 column using a gradient of 15% A to 55% B over 450 min (A=0.1% TFA, B=0.1% TFA/50% CH3CN). Five-minute fractions were collected while monitoring the U.V. at 214 nm (2.0A). The appropriate fractions were combined, frozen and lyophilized. MALDI-mass spectral analysis indicated a mass of 3313.85 g for the purified ghrelin, which was consistent with the theoretical molecular weight.

We tested mono-octanoylated ghrelin and tested for ability to release GH in a primary rat pituitary cell assay.

### EXAMPLE 2

### Animals

Wild-type mice (129SV strain) and NPY-knockout mice were obtained from Taconic Farms®. Eight-week old dwarf rats were purchased at Harlan UK. Animals were housed individually in a temperature controlled environment (25° C.) with a 12-hour light and 12-hour dark (18.00−06.00) photoperiod. All mice had ad libitum access to pelleted mouse food (5008 PMI® Nutrition International) and tap water. Mice were between 9 and 13 weeks of age and were injected daily between 17.00 and 18.00 with 0.1 ml of phosphate buffered saline (PBS) containing 0 or 8 mg/kg/d ghrelin over 13 days. Food intake and body weights were measured daily at 08.00 h. All animal experiments were conducted in accordance with the principles and procedures outlined in the National Institute of Health (NIH) Guide for the Care and Use of Laboratory Animals.

### EXAMPLE 3

### Indirect Calorimetry

Twenty-four hour energy expenditure (EE) and respiratory quotient (RQ) were measured by indirect calorimetry using an open circuit calorimetry system (Oxymax, Columbus Instruments International Corporation; Columbus, Ohio). The instrument was calibrated before each experiment using standard gas mixtures containing known concentrations of CO₂, N₂ and O₂. After the first daily injection, animals were placed in calorimeter chambers containing food and water in a room maintained under identical conditions as those described above throughout the treatment period. Gas sampled from each of 10 chambers was first dried by a condenser. The volume of oxygen consumed (VO₂) and carbon dioxide produced (VCO₂) in an hour was measured using a paramagnetic oxygen sensor and a spectrophotometric CO₂ sensor. Such measurements were obtained hourly for 24 hours. RQ is the ratio of VCO₂ to VO₂. EE was calculated as the product of calorific value of oxygen (CV) and VO₂ per kilogram (kg) of body weight; where CV=3.815+1.232*RQ (Elia, M. & Livesey, G, World Rev Nutr Diet 70, 68-131 (1992)). Total calories expended were calculated to determine daily fuel utilization. To calculate proportion of protein, fat and carbohydrate that is utilized during that 24-hour period, we used Flatt's proposal and assumed that protein utilization was equivalent to protein intake for adult stable animals (Flatt, J. P., J. Nutr Biochem 2, 193-202 (1991)). Using formulae and constants derived by Elia and Livesey (Elia, M. & Livesey, G., World Rev Nutr Diet, 70, 68-131 (1992)), we calculated the percent of daily fuel utilization derived from carbohydrate and fat. Daily caloric intake was calculated as (mass of daily food intake in g)*(physiological fuel value of the diet in kcal/g). Locomotor activity was measured by counting the number of times an animal breaks a new light beam during each of 24 hours in the calorimeter.

### EXAMPLE 4

### In-Vivo Analysis of Body Composition by Dual-Energy X-Ray Absorptiometry (DXA)

Body composition was measured on day 14 of the treatment period by DXA using a Norland p-DEXA® (Norland, USA). The system provides a non-invasive method for quantification of whole body composition and is based on the differential attenuation of high and low energy x-rays by the tissues in the scan area. Soft tissues attenuate the energy beam less than bone; of the soft tissue mass, fat tissue attenuates the beam less than lean tissue. Fat mass consists primarily of adipose tissue, but lean mass includes organs, tendons, cartilage, blood and body water in addition to skeletal muscle. In the present study, fat mass, lean mass and bone mineral content (bone mass) were measured and reported. Mice were anesthetized with inhalation of isoflorane and placed on the instrument platform in ventral position. Measurements were performed at a speed of 10 mm/min and a resolution of 0.5×0.5 mm. Quality controls using phantom ID2232 and Calibration Standard 82315 (Norland) were performed before starting measurements.

### EXAMPLE 5

### In Vivo Administration

Mice were treated with GHRP-2 for 18 days. A dose-dependent increase (n=42, p=0.001) in food intake and body weight was observed. A significant increase in fat mass (p=0.002) and bone mass (p=0.017) with no change in lean mass (p=0.63) was measured by dual-energy-X-ray-absorptiometry. This was partially a consequence of decreased (p=0.02) lipid utilization measured by indirect calorimetry. Hypothalamic mRNA levels (measured by RT-PCR) of neuropeptide Y (NPY), agouti-related-protein (AGRP), pro-opio-melanocortin (POMC) and melanocyte-concentrating hormone (MCH) were not changed. Since GHRP-6 increases c-fos expression in NPY-neurons (Vernon, R. G.; J Endocrinol 150, 129-40 (1996)) and because these neurons also release AGRP, a natural melanocyte stimulating hormone antagonist, GHRP-2 treatment was repeated in NPY-knockout mice (NPY-/−). Again, GHRP-2 induced a positive energy balance. However, an increase in AGRP mRNA levels (p=0.008, n=24 in GHRP-2 treated (NPY-/−)) was observed. Plasma levels of IGF-I, insulin, glucose and corticosterone were not changed. Thus, peripheral administration of GHRP-2 induces a positive energy balance and fat gain by a hypothalamic mechanism. 200 μg of rat ghrelin was injected subcutaneously into wild-type mice, GHRP-2 or vehicle (phosphate buffered saline). After 5 days of treatment, body weight increased (p=0.00) 12% in both ghrelin- and GHRP-2-treated mice but not in controls. This weight gain was a consequence of decreased energy expenditure and decreased lipid utilization. Similar data have been observed in hypophysectomized rats indicating that GH and the other pituitary hormones do not mediate this anabolic activity. Such data indicate that the new stomach hormone, ghrelin, is a powerful stimulator of caloric accretion and that hypersecretion of ghrelin creates an obese state.

### EXAMPLE 6

### Pituitary Cell Culture Assay for Growth Hormone Secretion

Thirty-two 250 g male Sprague-Dawley rats are used for each assay. The animals are killed by decapitation and anterior pituitaries are removed and placed into ice cold culture medium. The pituitaries are sectioned into eighths and enzymatically digested using trypsin (Sigma Chemical) to weaken connective tissue. Pituitary cells are dispersed by mechanical agitation, collected, pooled and then seeded into 24-well plates (300,000 cells/well). After 4 days of culture, the cells form an even monolayer. Cells are then washed with medium and challenged to secrete GH by the addition of varying log concentrations of grhelin and the test compound to the medium. After 15 min at 37° C., the medium is removed and stored frozen until standard radioimmunoassays for rat GH can be performed.

### EXAMPLE 7

### In Vitro Receptor Binding Assay

Recombinant CHO cells expressing the human growth hormone secretagogue receptor cDNA described by Howard et al., Science 273: 974-977 (1996) are grown and harvested in nutrient medium. Membrane preparations are then obtained by first washing the cells with PBS buffer, then twice washing with cold buffer (25 mM HEPES, 2 mM MgCl₂, 1 mM EDTA, 20 μg/ml Leupeptin, 1 mM PMSF, 2 μg/ml Aprotinin, 50 μg/ml Trypsin Inhibitor, pH 8.0) and resuspending in buffer. The cell suspension is lysed in a glass Teflon® homogenizer, and the resulting sample is then centrifuged at 35,300×g for 30 minutes at 4° C. The supernatant is removed, and the pellet is resuspended in cold buffer and homogenized. Aliquots may then be prepared and stored at −80° C.

A sample of the membrane preparation is pre-incubated with a test compound or a control compound with and without added grhelin in buffer (25 mM HEPES, 0.2% (w/v) BSA, pH 7.6) at 32° C. for 10 minutes. Reaction buffer (final concentration: 25 mM HEPES, 0.2% (w/v) BSA, 2.6 mM Mg, 0.8 mM ATP, 0.1 mM GTP, 5 mM creatine phosphate, creatine kinase 50 U/ml, 0.2 mM IBMX, pH 7.6) is added and incubated for an additional 30 minutes. Incubations are stopped by adding 10 mM EDTA.

Production of cAMP is assayed using a fluorescent tracer-immuno assay method. In brief, after the incubation is stopped, fluorescent tracer (cAMP-b phycoerythrin conjugate) is added followed by the addition of affinity purified anti-cAMP rabbit antiserum. After incubation at room temperature for 45 minutes, anti-rabbit IgG coated assay beads are added and incubated for an additional 15 minutes. Plates are then evacuated and read on a Pandex® PFCIA reader.

In this assay, ghrelin binding shows decreasing fluorescent intensity due to increased cAMP concentration. Fluorescent intensity values are correlated to rate of cAMP production (pmol/min/mg). Conversely, inhibition of ghrelin binding by either receptor blockade or ghrelin neutralizations shows no decrease in fluorescent intensity.

### EXAMPLE 8

### Rat Ghrelin Response to Fasting and Refeeding

Because ghrelin is mainly generated by the stomach and secreted into circulation, we measured plasma ghrelin levels by radioimmunoassay. Elevated ghrelin levels after fasting in 250 g male Sprague-Dawley rats (p=0.001) were decreased to normal levels (1.3±0.1 ng/ml) by re-feeding normal rat chow or by oral gavage of dextrose (p=0.001), but not after stomach expansion with water.

### EXAMPLE 9

### Human Ghrelin Response to Glucose Ingestion

Plasma ghrelin concentrations were measured in 5 women (BMI 23.5±3.3 kg/m2, body fat 23±35%) over 24 hours during which 3 meals (total energy=1795±105 kcal) containing 55/30/15% of energy as carbohydrate, fat, and protein, respectively were consumed. When 30% of energy was in the form of a glucose-sweetened beverage, plasma ghrelin decreased by 30% 2 hours after the meal (p<0.01), and over the 24 h sampling period (p<0.05). In contrast, when a fructose-containing beverage was consumed with each meal resulting in reduced postprandial glucose and insulin excursions, plasma ghrelin levels did not decrease after meals. We conclude that glucose ingestion and (or) the resulting insulin response appear to be candidates regulating ghrelin secretion. Further, we speculate that ghrelin release is a normal response to fasting. Such elevated ghrelin stimulates appetite and the utilization of carbohydrate (determined in above examples using rodents) and thus corrects hypoglycemia resulting from fasting. Ingestion of glucose rescues hypogylcemia and thus inhibits ghrelin secretion from the stomach to prevent hyperglycemia. Thus, ghrelin plays an important role in the regulation of blood glucose. Agents that block ghrelin action may be useful for the treatment of diabetes.

1-17. (canceled)

18. A method of assaying a compound for activity as a ghrelin neutralizing agent (GNA) comprising:
a) preparing a mixture of the compound, and ghrelin; allowing said mixture to incubate for a period of time under conditions sufficient to permit binding;
b) adding isolated pituitary cells to said mixture; and,
c) measuring the release of growth hormone after a period of time.

19. The method of claim 18, wherein the compound is selected from the group consisting of an antibody, an antibody fragment, and a single chain antibody.

20. A method of assaying a compound for activity as a ghrelin neutralizing agent (GNA) comprising:
a) preparing a mixture of the compound and ghrelin;
b) allowing said mixture to incubate for a period of time under conditions sufficient to permit binding;
c) adding isolated pituitary cells to said mixture;
d) assaying for levels of cAMP; and
e) comparing the level of cAMP to control levels.

21. The method of claim 20, wherein the compound is selected from the group consisting of an antibody, an antibody fragment, and a single chain antibody.

22. An in vivo method of assaying a compound for activity as a GNA comprising:
a) optionally fasting a rodent for at least 24 hours;
b) dosing the rodent with the compound;
c) allowing the rodent to eat ad libitum for at least 24 hours; and,
d) comparing fat deposition, food intake, energy expenditure, and/or respiratory quotient to control rodents.

23. The method of claim 22, wherein the compound is selected from the group consisting of an antibody, an antibody fragment, and a single chain antibody.
